(19) United States
(12) Patent Application Publication
(10) Pub. No.: US 2026/0081017 A1
(43) Pub. Date: Mar. 19, 2026

(54) SYSTEMS AND METHODS OF CREATING, TRAINING, AND DEPLOYING AN AI-BASED ADVERSE EVENT PREDICTION MODEL AND PLATFORM

(71) Applicant: Darroch Medical Solutions, Inc., San Diego, CA (US)

(72) Inventors: Anthony Shao, Hillsborough, CA (US); Colby Bishop, San Diego, CA (US); An Tran, San Diego, CA (US); Tanny Henry, San Diego, CA (US)

(21) Appl. No.: 18/884,178

(22) Filed: Sep. 13, 2024

(51) Int. Cl. G16H 50/20; G16H 40/63; G16H 50/30

(52) U.S. Cl. CPC G16H 50/20; G16H 40/63; G16H 50/30

(57) ABSTRACT

Methods and systems of creating artificial intelligence processes for predicting adverse events in a patient are provided in which processed vital signs medical data are extracted from a server in communication with one or more vital signs monitors, and raw non-vital signs medical data are extracted from one or more medical instruments, including extracting auxiliary medical data which are not displayed by the medical instruments. The processed vital signs medical data and raw non-vital signs medical data are sent to a cloud-based artificial intelligence platform, which inputs the combined medical data into an AI-driven adverse event prediction model. The adverse event prediction model is continually and automatically fed the combined medical data. The adverse event prediction model identifies adverse event precursors and provides an assessment of a level of risk that a patient will experience an adverse event and a timeframe within which the adverse event is likely to occur.

### FIELD OF THE DISCLOSURE

The present disclosure relates to systems and methods of creating an artificial intelligence process for predicting adverse events in a patient. The present disclosure relates to combined systems and methods of extracting medical data to develop an AI-based adverse event prediction model and predicting adverse events in patients.

### BACKGROUND

Adverse events (AEs) are defined as unintended injuries, complications, or otherwise negative outcomes associated with medical care. AEs that occur with treatment can include medication side effects, injury, psychological harm or trauma, and death and often have a wide range of severity. In a study by McDermott et al. (2017), one in three patients experienced an AE during their stay in a hospital; other studies have suggested that more than one in ten patients will experience an AE during their stay. It is estimated that adverse events and medical errors annually affect 250,000+ patients, lead to 100,000 deaths, and cost anywhere between $17B to $29B with many believing that these numbers may be underreported due to documentation discrepancies.

Recent studies have suggested that a significant number of AEs can be prevented with improved patient monitoring and recognition of AE precursors. In many cases, unusual fluctuations in a patient's vital signs, and by extension a patient's medical instrument data, can serve as an indication of an impending adverse event, as AE precursors and early signs can be observable hours or even days before an event. However, current medical instrument and monitoring technologies are not equipped to address the fast-moving nature of AEs, resulting in AEs being commonly reacted to at onset rather than actively anticipated or prevented.

The genesis of this issue primarily concerns data aggregation. At present, the most common form of data aggregation systems are Electronic Medical Records (EMRs), nursing stations and other computerized monitoring systems; in most cases, these systems typically only receive data that is manually entered or batch-processed via a medical instrument server a few times a day, with the manually entered data having a high likelihood of transcription errors. In most cases, incoming data to these computerized systems is reserved for intermittent vital signs, updated labs, or additional demographic information.

Unfortunately, early-stage AE precursors are often subtle and do not trigger medical instrument alarms; they are often reflected in minute changes in a patient's medical instrument data and require continuous data aggregation to capture and understand. Thus, if an AE precursor occurs between the manual entry or batch-processed data input into EMRs, there is an extremely high likelihood that it will be missed, and the patient will likely incur worsening symptoms until a practitioner notices or the condition becomes severe enough to trigger a medical instrument alarm.

Worse still, there does not appear to be a universally agreed data protocol that can account for the differing data structures between both medical instruments and medical instrument vendors, and therefore, continuous data from other medical instruments (e.g., infusion pumps and beds) is not continuously captured, despite their potential value in AE prevention. Practitioners always have the option to manually assess the patient to determine AE precursors, but this approach is challenging at best due to workflow issues, overwhelming workloads, demanding patient-to-practitioner ratios, and increasing staff shortages; in addition, physically assessing a medical instrument at a specific point in time only reflects an instantaneous measurement. Context, data history, and trends are all critical components of determining an AE precursor and its likelihood of occurrence.

Various technologies have attempted to address the data issues. Companies have attempted to create and implement smart, IoT devices that can stream data to EMRs at high frequencies, but EMRs themselves were not designed to house high frequency data, and thus the majority of useful information remains idle in vendor-specific servers. Older devices often do not have any form of interoperability, and thus do not have any ability to transmit to another computerized data system. While there are new medical instruments that may have the ability to transmit some or all of their data, many of the systems are cost-prohibitive to implement and therefore are not used. Thus, from a data perspective, data entry into computerized systems remains in two forms: manual transcription, which is prone to transcription mistakes, or batch-process of data, which may only occur a few times a day.

Various academic institutions have begun implementing EMR-based AI algorithms to aid in AE prevention, primarily in sepsis and hospital-acquired infections. Unfortunately, the vast majority of these machine learning and AI models have fallen short of expectations due to a lack of robust datasets needed to train, improve, and achieve a level of model accuracy required for a hospital's standard of care. Almost all training datasets are derived from EMRs or publicly available databases (e.g., MIMIC-IV), which are littered with transcription and timing errors, missing and incorrect values, and poor data consistency. As a result, data scientists must impute or carry forward existing data values, which can simplify a dataset for training but leads to models that oversimplify complex patterns or fail to capture important nuances in the data. Due to the irregular and random intervals of data collection in medical settings, models must be trained on data resampled to consistent time increments or be manually adjusted (perhaps incorrectly) to fit.

FIG. 1 shows several of the issues present with EMR-based datasets. Chart time refers to the time in which the values were taken; the time between the first and second reading is roughly two hours, and the time between the second and third reading is almost six hours; these times are also shifted randomly into the future in order to de-identify patient datasets. In most current technologies, the frequency of data aggregation is dependent on the final computerized system, medical instrument server, or network structure, all of which can severely reduce the frequency of data outgoing to a medical instrument server and nurse.

Readings one and two have typical metric values, but reading three only has pulse oxygen. Reading seven has a relatively unlikely value of 10% for pulse oxygen, to which reading does not occur until eight hours later. Significant data wrangling must be done in order to make these datasets useful for AI training, but in doing so, data scientists may incorrectly “correct” a value in order to make it fit into the overall dataset.

Upon model deployment, there are no pre-processing algorithms that can easily rectify transcription mistakes or batch-processed data. A model will not inherently know if a reading, such as 10% pulse oxygen, is physiologically plausible or erroneous, and it will generate an output based on the input it receives. This often leads to false positives and negatives, in which the latter are much more potentially damaging to the patient. Models must also be deployed within EMRs to obtain the same data the model was trained on; if the data point does not come through at the correct interval, a model can either attempt to handle the missing value and potentially lower its performance characteristics, or simply fail to generate an output for that time point. While AI has the potential to revolutionize how AEs and their precursors are identified, data quality and consistency issues are quickly showing why current AI algorithms cannot represent the changes a patient undergoes.

Thus, there is a need for a solution that can act as a vertically integrated data aggregation, analysis, and presentation system. This system must also have the ability to integrate with existing instruments. For AI to truly bridge the acuity gap, there is also a need for data infrastructure distinct from EMRs. In addition, there is a need for an AI system that has the freedom to obtain data beyond vital signs; data from other bedside medical instruments should be considered, otherwise certain AEs such as adverse drug events, IV infiltration and/or extravasation, and bed falls will remain “unpreventable” adverse events.

### SUMMARY

The present disclosure, in its many embodiments, alleviates to a great extent the disadvantages of known AE prevention systems by providing an AI-driven continuous health status monitoring and AE prevention platform that offers high frequency, real time monitoring of any type of medical instrument, monitor, or medical patient data metrics, including but not limited to, vital signs, infusion pumps, and in-bed activity. By leveraging and cross correlating minute-to-minute (or more frequent) data metrics from multiple medical instruments via cloud-based machine intelligence engines, disclosed systems and methods can create patient-specific, actionable insights that alert practitioners to certain AE precursors and give legitimate timeframes for potential AE onset. Disclosed systems and methods create better knowledge workers and help healthcare systems curb unnecessary costs associated with unintended injuries.

It is an object of the disclosure to extract data from multiple sources, but continuous metric and sensor data from two of three sources is not available at the moment. At a high level, disclosed methods and systems accomplish the following: the collection of data and metadata from multiple distinct instruments which yields new features; creation of new models with these features; deployment of the models on the same data aggregation subarchitecture for model efficacy. This overcomes a significant limitation of current systems, namely, the reliance on manual data transcription. Such manual processes introduce uncertainty regarding the timing of data availability and the accuracy of the data itself, thereby compromising the model's ability to operate with consistent and reliable inputs.

An exemplary computer-implemented method of creating an artificial intelligence process for predicting adverse events in a patient comprises extracting processed vital signs medical data from a server in communication with at least one vital signs monitor and extracting raw non-vital signs medical data from one or more medical instruments, including extracting auxiliary medical data, e.g., metadata and/or alarm conditions, which are not displayed by the one or more medical instruments by identifying analog sensor data and deconstructing digitized communication packets. The processed vital signs medical data and the raw non-vital signs medical data are combined and provided to an artificial intelligence platform to train an AI-driven adverse event prediction model on the combined processed vital signs medical data and raw non-vital signs medical data. The AI-driven adverse event prediction model generates novel features and composite features from the combined processed vital signs medical data and raw non-vital signs medical data to identify patterns indicative of potential adverse events. The artificial intelligence platform may be cloud-based.

Exemplary methods include the steps of continuing to extract processed vital signs medical data from the server and continuing to extract raw non-vital signs medical data from the one or more medical instruments, including continuing to extract the auxiliary medical data which are not displayed by the one or more medical instruments by identifying analog sensor data and deconstructing digitized communication packets. The processed vital signs medical data and the raw non-vital signs medical data are combined and automatically provided to the artificial intelligence platform to feed into the AI-driven adverse event prediction model. The AI-driven adverse event prediction model employs machine learning techniques to continuously improve predictive accuracy based on new input data and outcome feedback.

The AI-driven adverse event prediction model analyzes the combined processed vital signs medical data and raw non-vital signs medical data for potential onset of at least one adverse event including identifying adverse event precursors in the combined processed vital signs medical data and raw non-vital signs medical data. The AI-driven adverse event prediction model provides an assessment of a level of risk that a patient will experience an adverse event and a timeframe within which the patient will experience the adverse event. Exemplary methods further comprise tuning the adverse event prediction model. The tuning may include modifying data frequency characteristics, adjusting a risk threshold, and/or modifying a lookback window and a prediction window, which in turn update the sensitivity and specificity of the model.

In exemplary embodiments, extracting raw non-vital signs medical data from one or more medical instruments, including extracting auxiliary medical data, e.g., metadata and/or alarm conditions, which are not displayed by the one or more medical instruments, generates a continual stream of the raw non-vital signs medical data from one or more medical instruments that otherwise do not have an independent ability to continually stream the raw non-vital signs medical data.

Exemplary embodiments of an artificial intelligence system for predicting adverse events in patients comprise at least one dongle, a microcomputer, a cloud database, and an artificial intelligence platform. The dongle is configured to attach to at least one medical instrument to extract raw non-vital signs medical data from the medical instrument, including extracting auxiliary medical data which are not displayed by the at least one medical instrument by identifying analog sensor data and deconstructing digitized communication packets. The microcomputer is in communication with the at least one dongle and receives the raw non-vital signs medical data from the dongle.

The cloud database is in communication with the microcomputer and a client server. The client server receives the processed vital signs medical data from at least one vital signs monitor, and the cloud database receives the raw non-vital signs medical data from the microcomputer and the processed vital signs medical data from the client server. An artificial intelligence platform is in communication with the cloud database. The artificial intelligence platform receives the raw non-vital signs medical data and the processed vital signs medical data from the cloud database. The artificial intelligence platform inputs the raw non-vital signs medical data and the processed vital signs medical data into an AI-driven adverse event prediction model. The AI-driven adverse event prediction model generates novel features and composite features from the the processed vital signs medical data and the raw non-vital signs medical data to identify patterns indicative of potential adverse events.

In exemplary embodiments the medical instrument is a load sensor or set of load sensors in/for a patient's bed, and the processed vital signs medical data are pulse rate, pulse rate variability, respiratory rate, blood pressure, and blood oxygen. In exemplary embodiments, the medical instrument is an infusion pump and the raw non-vital signs medical data include backflow pressure. The dongle generates a continual stream of the raw non-vital signs medical data from the at least one medical instrument that otherwise does not have an independent ability to continually stream the raw non-vital signs medical data.

In exemplary embodiments, the dongle continues to extract raw non-vital signs medical data from the one or more medical instruments, including continuing to extract the auxiliary medical data which are not displayed by the one or more medical instruments. The microcomputer continues to receive the raw non-vital signs medical data from the dongle. The client server continues to receive the processed vital signs medical data from the at least one vital signs monitor, and the cloud database continues to receive the raw non-vital signs medical data from the microcomputer and the processed vital signs medical data from the client server. The artificial intelligence platform continues to receive the raw non-vital signs medical data and the processed vital signs medical data from the cloud database.

The artificial intelligence platform automatically inputs the raw non-vital signs medical data and the processed vital signs medical data into an AI-driven adverse event prediction model. The AI-driven adverse event prediction model employs machine learning techniques to continuously improve predictive accuracy based on new input data and outcome feedback. In exemplary embodiments, the AI-driven adverse event prediction model also generates risk scores for potential adverse events based on the identified patterns and relationships and continuously updates and refines its predictive capabilities through iterative learning from new data inputs and feedback.

In exemplary embodiments, the AI-driven adverse event prediction model analyzes the processed vital signs medical data and raw non-vital signs medical data for potential onset of at least one adverse event, including identifying adverse event precursors in the processed vital signs medical data and the raw non-vital signs medical data, and provides an assessment of a level of risk that a patient will experience an adverse event and a timeframe within which the patient will experience the adverse event.

An exemplary combined system of extracting medical data to train an AI-based adverse event prediction model and predicting adverse events in patients comprises at least one dongle, a microcomputer, a cloud database, and an artificial intelligence platform. The dongle is configured to attach to at least one medical instrument such that the dongle extracts raw non-vital signs medical data from the medical instrument, including extracting auxiliary medical data which are not displayed by the at least one medical instrument by identifying analog sensor data and deconstructing digitized communication packets. The microcomputer is in communication with the dongle and receives the raw non-vital signs medical data from the dongle.

In exemplary embodiments, the artificial intelligence platform is located in the cloud. The cloud database is in communication with the microcomputer and a client server. The client server receives processed vital signs medical data from at least one vital signs monitor, and the cloud database receives the raw non-vital signs medical data from the microcomputer and the processed vital signs medical data from the client server. The artificial intelligence platform is in communication with the cloud database. The artificial intelligence platform receives the raw non-vital signs medical data and the processed vital signs medical data from the cloud database and inputs the raw non-vital signs medical data and the processed vital signs medical data into an AI-driven adverse event prediction model. The AI-driven adverse event prediction model generates novel features and composite features from the the processed vital signs medical data and the raw non-vital signs medical data to identify patterns indicative of potential adverse events.

The dongle continues to extract the raw non-vital signs medical data from the at least one medical instrument, including continuing to extract the auxiliary medical data which are not displayed by the at least one medical instrument. In exemplary embodiments, the client server continues to receive processed vital signs medical data from the at least one vital signs monitor, and the cloud database continues to receive the raw non-vital signs medical data from the microcomputer and the processed vital signs medical data from the client server. The artificial intelligence platform continues to receive the raw non-vital signs medical data and the processed vital signs medical data from the cloud database.

The artificial intelligence platform automatically inputs the raw non-vital signs medical data and the processed vital signs medical data into an AI-driven adverse event prediction model. The AI-driven adverse event prediction model employs machine learning techniques to continuously improve predictive accuracy based on new input data and outcome feedback. In exemplary embodiments, the AI-driven adverse event prediction model generates real-time risk assessments for potential adverse events, provides explanations for its predictions, and adapts its predictive models based on feedback and new data to improve performance over time.

In exemplary embodiments, the AI-driven adverse event prediction model analyzes the processed vital signs medical data and the raw non-vital signs medical data for potential onset of at least one adverse event including identifying adverse event precursors in the combined processed vital signs medical data and the raw non-vital signs medical data and provides an assessment of a level of risk that a patient will experience an adverse event. In exemplary embodiments, the system further comprises a user interface whereby a medical practitioner can tune the adverse event prediction model. The tuning may include modifying data frequency characteristics, adjusting a risk threshold, and/or modifying a lookback window and a prediction window, which in turn update the sensitivity and specificity of the model.

Accordingly, it is seen that systems and methods of training artificial intelligence processes for predicting adverse events in a patient and combined systems and methods of extracting medical data to train an AI-based adverse event prediction model and predicting adverse events in patients are provided. These and other features and advantages will be appreciated from review of the following detailed description, along with the accompanying figures in which like reference numbers refer to like parts throughout.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 is a chart showing an example of an EMR-based dataset;

FIG. 2 is a schematic of an exemplary embodiment of a system for extracting medical data to train an AI-based adverse event prediction model and predicting adverse events in patients in accordance with the present disclosure;

FIG. 3 is a flow diagram of an exemplary system and method of extracting medical data from a medical instrument to train an AI-based adverse event prediction model in accordance with the present disclosure;

FIG. 4 is a perspective view of an exemplary embodiment of a hardware attachment, or dongle, in accordance with the present disclosure;

FIG. 5 is a flow diagram of an exemplary embodiment of a method of extracting medical data to input to an AI-based adverse event prediction model and predicting adverse events in patients in accordance with the present disclosure;

FIG. 6 is a flow diagram of an exemplary system and method of extracting medical data from a medical instrument to train an AI-based adverse event prediction model to detect downstream occlusions in peripheral venous catheter (PVC) systems in accordance with the present disclosure;

FIG. 7 is a front view of a user interface on a mobile device screen showing an exemplary embodiment of an AI platform in accordance with the present disclosure;

FIG. 8 is a front view of a user interface on a mobile device screen showing an exemplary embodiment of an adverse event prediction model in accordance with the present disclosure; and

FIG. 9 is a front view of a user interface on a mobile device screen showing an exemplary embodiment of an AI platform with options for tuning performance of an adverse event prediction model in accordance with the present disclosure.

### DETAILED DESCRIPTION

In the following detailed description of exemplary embodiments of the disclosure, reference is made to the accompanying drawings in which like reference numbers indicate similar elements, and in which is shown by way of illustration specific embodiments in which disclosed devices, systems, and methods may be practiced. These embodiments are described in sufficient detail to enable those skilled in the art to practice the embodiments, and it is to be understood that other embodiments may be utilized, and that logical, mechanical, functional, and other changes may be made without departing from the scope of the present disclosure. The following detailed description is, therefore, not to be taken in a limiting sense, and the scope of the present disclosure is defined by the appended claims. As used in the present disclosure, the term “or” shall be understood to be defined as a logical disjunction and shall not indicate an exclusive disjunction.

A high level description of the data flow (described in detail herein) of exemplary embodiments is as follows. Data from an infusion pump and/or bed are sent to a microcontroller and subsequently to a cloud database. Data from a vital signs monitor is sent to a client server, and the client server sends the data to the cloud database. In exemplary embodiments, the cloud AI platform is basically a virtual machine that hosts the already created models and receives data from the cloud database at set intervals. Since the cloud AI platform hosts the already-created models, any changes from the user regarding tuning of the model are made in the cloud AI platform. The cloud AI platform then analyzes the received datasets and then pushes the output either to the cloud database and then to the adverse event prediction model.

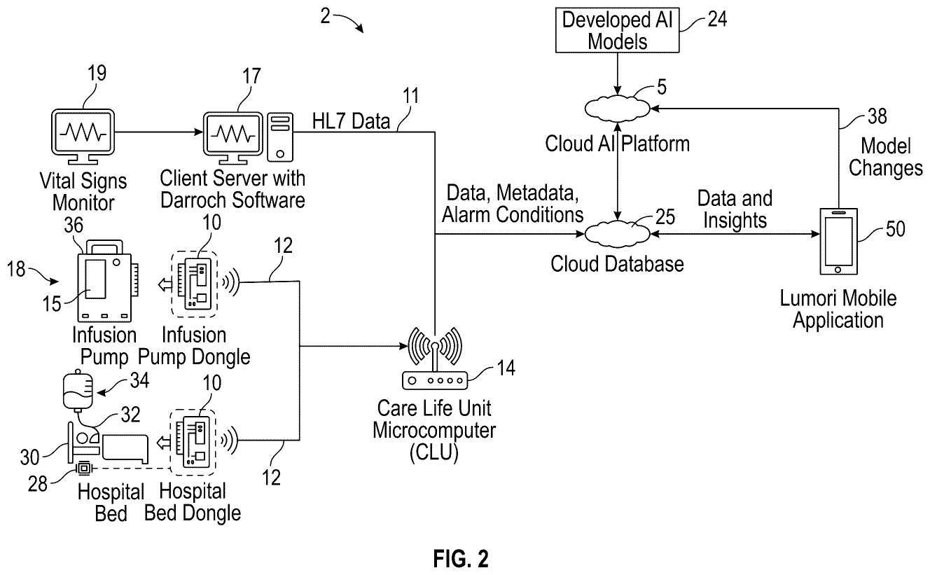

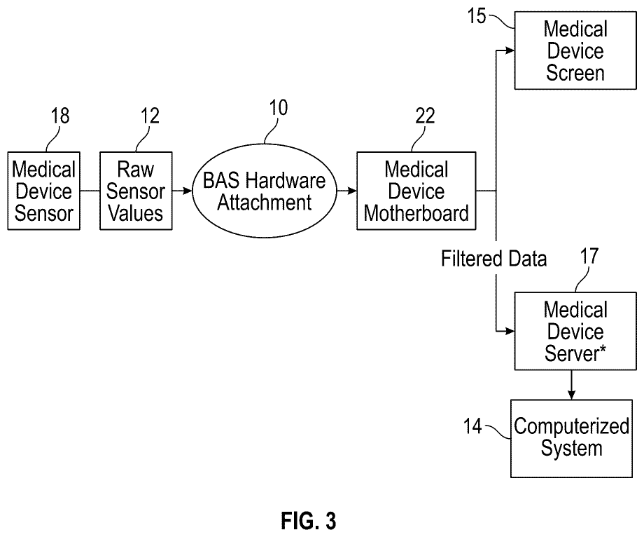

As shown in FIGS. 2 and 3, disclosed systems 2 and methods include AI-driven, autonomous, continuous health status monitoring and AE prevention platforms 5 that provide high frequency, real time data aggregation, analysis, and presentation of vital signs, infusion pumps, and in-bed activity. An exemplary method 1 of training an artificial intelligence process for predicting adverse events in a patient starts by extracting and aggregating medical data from at least two sources or types of sources. The first is a conventional source of processed vital signs medical data 11 such as a medical instrument server 17 and/or the observation (OBX) fields of the common HL7 transmission protocol, and/or a vital signs monitor 19.

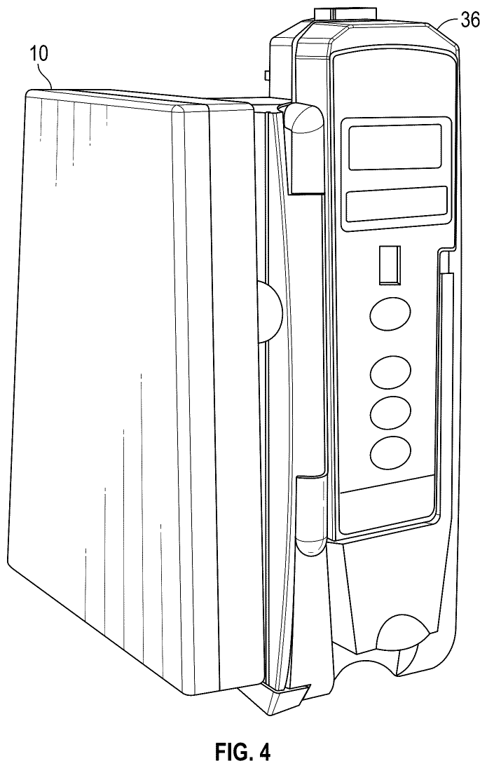

The second type of source is existing medical instruments 18, from which disclosed embodiments can extract raw non-vital signs medical data 12 via proprietary hardware connections, or dongles 10. An exemplary dongle 10 is shown in FIG. 4 connected to an infusion pump 36. In exemplary systems 2, each medical instrument has a specially developed hardware connection 10 that passively monitors and extracts the medical instrument's data and metadata at a uniform frequency. Advantageously, the dongles 10 allow for the extraction of metrics that are outside of common transmission protocols such as FHIR's HL7 protocol; the OBX fields of HL7 are almost always reserved for only vital signs. The dongle 10 may have a synchronous input/output (I/O) unit and an asynchronous I/O unit and be configured to route through either.

In exemplary embodiments, the raw non-vital signs medical data 12 are extracted from the medical instruments 18 in real time. The dongles 10 enable extraction of auxiliary medical data such as metadata and alarm conditions which are not displayed by the medical instruments 18 and therefore would not otherwise be accessible to medical practitioners. Moreover, without the dongles 10, a significant amount of valuable information remains on the medical instrument 18 unaggregated, thereby limiting the number and quality of features an AI process may incorporate. The dongles 10 generate a continual stream of raw non-vital signs medical data 12, which the medical instruments 18 otherwise would not have the ability to stream.

Implementing a hardware-based passthrough such as dongle 10 allows for two important improvements over current technologies. First, connecting to a medical instrument 18 directly removes the randomness of manual entry of data into computerized systems and allows a distinct system to control the frequency of data aggregation. Second, the implementation of hardware attachments such as the dongles 10 before the medical instrument's server 17 allows for the extraction and aggregation of certain data and metadata that are not presented on the medical instrument's screen 15 or sent out via its server 17. A third benefit is that data can be aggregated on a much faster scale, e.g., up to 10 readings/sample or second. In any hospital, that would be too much data to put onto the network, and it would be absolutely infeasible to expect nurses to be able to write this much data down. By using the dongles 10, the system 2 can access, aggregate, and save all this information without significantly overburdening the network (through the use of Bluetooth communications between dongle 10 and microcomputer 14).

FIGS. 2, 3 and 4 show a typical implementation location of a hardware attachment 10. From a broad perspective, the hardware-based approach of the present disclosure targeting specific sensors before the analog to digital converter in the medical instrument's motherboard allows the hardware attachment 10 to dictate the data aggregation frequency and access data outside of common transmission protocols such as FHIR's HL7 protocol. In exemplary embodiments, the hardware attachment 10 is typically between the sensors and the motherboard and passively monitors the analog output of the sensors. Using its own analog to digital converter, communication packets are deconstructed to determine what kind of data and/or metadata are passed and if any alarm conditions are being triggered.

In exemplary embodiments, the dongles 10 are controlled via a microcomputer 14 within the patient's room. This provides for two-way communication between the dongle 10 and the microcomputer 14, which allows for changes within the hardware attachments'10 data aggregation frequency 56, as well as continuous data capture without significant strain on the network. These hardware connections then each send their data to microcomputer 14, which acts as the patient's local network hub 16, and which may be located in the patient's room via some form of communication. In exemplary embodiments, communication is in the form of Bluetooth or some derivation of Bluetooth. The local network hub 16 takes and secures these raw data streams and passes them to the cloud database 25 and then to the AI platform 5. This is the data aggregation subsystem which does not rely on manual transcription, batch-processing of data, and EMRs; advantageously, it is its own distinct data aggregation system.

Utilizing the cloud 20 for the AI platform 5 for data analysis yields several advantages. Developed models can quickly analyze large swaths of data and cross-correlate data from distinct medical instruments 18 to identify AE precursors. This is not possible in existing EMR-based systems, as EMRs do not receive the proper data frequency nor do they have the ability to acquire data from multiple medical instruments 18. Leveraging the cloud 20 also enables multi-AE analysis rather than a single model performing AE analysis serially. As discussed herein, because the data aggregation subsystem is vertically integrated into disclosed systems and methods as a whole, models will see the same data that they were trained on. Models will not be expecting a certain level of information and then not receive it because the data is manually inputted; all data aggregation and analysis is carried out together so as to ensure the models perform as autonomously and correctly as possible.

The processed vital signs medical data 11 and raw non-vital signs medical data 12 are combined, provided to an AI platform 5 via the cloud database 25, and used to train an adverse event prediction model 24. More particularly, aggregated metrics from the medical instrument server 17 or similar and the dongles 10 are used to create AI processes that target specific AEs. It should be noted that the effective models created by disclosed embodiments are novel; many of these AEs currently do not have reasonable prevention models. As discussed in more detail in connection with some of the examples, these system-specific processes consider multiple device metrics that cannot possibly be created or replicated without using the metrics gathered by the disclosed hardware attachments.

For the adverse event prediction model 24 to work, it must see the same data as it was trained on at the same frequency. Having a completely vertically integrated data extraction, analysis, and presentation system allows for optimized AI outputs 26 that are more visible and understandable for the healthcare practitioner. In turn, this may further reduce the incidence of AEs, as practitioners will be able to understand what the model specifies as the most important factor in an AE's precursor, as well as provide practitioners with the ability to potentially change how the model 24 performs. This is not possible in current systems as all data systems today are too fragmented to work together, do not consider data outside of EMRs, which by extension are predicated on manual transcription, and rely too heavily on subjective input. Exemplary embodiments ensure that developed AE prevention models 24 see the same data that the model was trained on; this is the best way to ensure model efficacy and value.

Thus, after building the AE prevention model 24 disclosed systems and methods continue to extract the variety of processed vital signs medical data 11 from the medical instrument server 17, HL7® protocol, or similar and the raw non-vital signs medical data 12 (e.g., in the form of metadata) from the medical instruments 18 in real time. The raw non-vital signs medical data are sent to the local network hub 16 and then to the cloud database 25. The vital signs medical data 11 are sent to the client server 17. The server 17 processes the vital signs medical data 11 and sends them to the cloud database 25. There, the processed vital signs medical data 11 and raw non-vital signs medical data 12 are combined and input into the AI-driven adverse event prediction model 24.

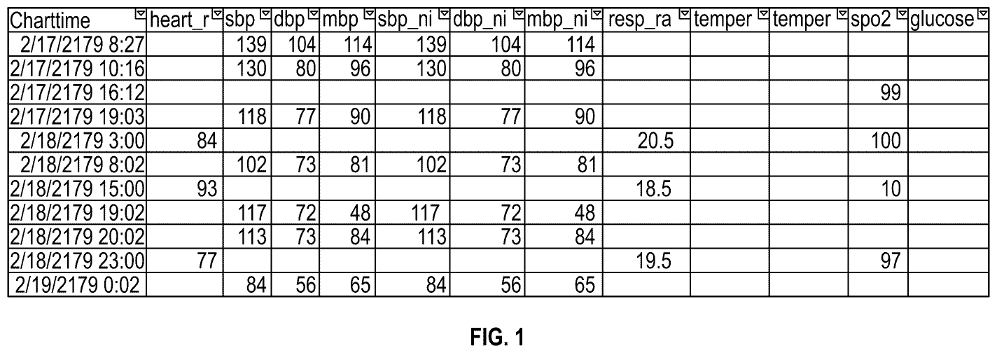

Advantageously, this process of data extraction, juxtaposition, and streaming is continual and automatic. This is also important because it maximizes the potential of the AE prediction model 24 by ensuring that it continues to receive the same types of data it has been trained to use. It should be noted that the reason most AI models perform poorly is that the data fed into them is manually inputted from nurses. That is, while current AI models are supposed to receive the data they are trained on, they often receive inaccurate data due to manual entry into EMRs. An example of the type of results a current model might produce is shown in FIG. 1. The present systems and methods advantageously remove the randomness associated with manual input by automating the entire process, which means providing accurate values and timestamps.

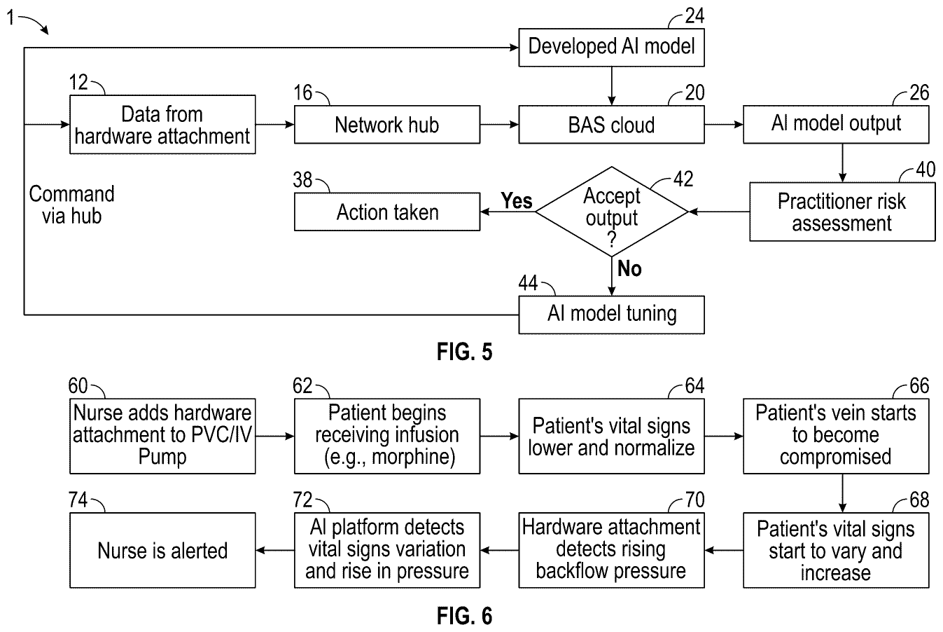

Referring to FIGS. 2 and 5, in exemplary embodiments a dongle 10 is connected to load sensor 28 for the bed 30 of a patient in a medical facility to collect raw data 11 from the load sensor 28. In this example, the dongle 10 extracts digitized load sensor values before they are processed by the bed's motherboard 22. As illustrated in FIG. 5, those values are sent to the patient's local network hub 16. Processed vital signs medical data 11 for the patient in the bed 30 are extracted from a vital signs monitor 19 or similar and also sent to the server 17. These data 11 might include pulse rate, pulse rate variability, respiratory rate, blood pressure, and/or blood oxygen. Both sets of data 11, 12 are sent to the cloud database 25, juxtaposed, and input into the adverse event prediction model 24, which uses the data to derive novel features that target the restlessness exhibited by the patient prior to a potential exit.

To derive these novel features, the adverse event prediction model 24 analyzes the patterns and correlations between the load sensor data and the vital signs data. The model 24 utilizes temporal pattern recognition to identify specific movement signatures, such as the frequency and amplitude of weight shifts or sudden changes in weight distribution that could indicate attempts by a patient to sit up. These movement patterns are then correlated with changes in vital signs; for instance, decreased heart rate variability combined with more frequent weight shifts might suggest growing anxiety or discomfort.

In exemplary embodiments, the adverse event prediction model 24 also creates composite features that integrate data from multiple sources, such as a “restlessness score” that factors in movement frequency, vital sign changes, volume of IV fluid infused, and time of day. Furthermore, the model 24 may employ anomaly detection to identify unusual patterns that deviate from a patient's baseline, such as unexpected correlations between vital signs and movement. These derived features collectively enhance the prediction accuracy of potential adverse events, enabling timely interventions. Importantly, the adverse event prediction model 24 continuously refines these features based on ongoing data input and feedback from healthcare providers, thereby improving its predictive capabilities over time.

Turning again to FIG. 2 and to FIG. 6, another exemplary case of correlating processed vital signs metrics and raw non-vital signs metrics will now be described. Peripheral venous catheters (PVC) 32 are one of the most frequently used medical instruments in hospital settings and are used to deliver fluid or drugs to a patient. Downstream occlusions (blockages or resistance at the level of the PVC insertion) are fairly common in patients who receive a PVC 32 and are often the result of a vein hardening due to external fluid being pushed into the vein itself. If left untreated, the resulting pressure that the vein exerts on the PVC line can cause the PVC 32 to, in turn, increase the pressure to deliver the therapy to the patient. This causes a feedback loop which can cause the vein to burst, leading to IV infiltration and/or extravasation, the patient to not receive a potentially critical infusion, and much more serious adverse events.

PVC systems 34 measure the backflow pressure that the vein exerts on the PVC line. This is how they determine how “hard” to pump fluid into the patient. In most cases, this value is only used to alert the practitioner if a certain pressure threshold is met, to which the medical instrument will sound 74 an alarm (i.e., downstream occlusion alarm). The present system extracts and aggregates IV pump metrics continuously. More particularly, backpressure data from an IV pump 36 may be extracted as metadata using a dongle 10 attached 60 by the practitioner to the IV pump, as well as volume-to-be-infused (VTBI) data. Many of these metadata features are not shown on the IV pump 36 but have significant use cases in AE prevention.

By aggregating these metrics continuously, the system 2 can identify trends in the backflow pressure which are consistent with occlusion precursors (e.g., rising backflow pressure). At the same time, processed vital signs medical data 11 for the patient receiving 62 the IV fluids or medication are extracted from the client server 17 or similar and sent to the local network hub 16. These data could include pulse rate, pulse rate variability, respiratory rate, blood pressure, blood oxygen, and/or temperature. The raw IV pump data 12 and processed vital signs data 11 are sent to the cloud database 25, juxtaposed, and input into the adverse event prediction model 24. By extracting and aggregating the two types of data 11, 12 continuously, and correlating backflow pressure with vital signs, a specific model can be created to determine the relationship between the drug being infused and the physiological response through vital signs.

When the patient begins receiving 62 an infusion, the patient's vital signs initially lower and normalize 64. However, as discussed above, downstream occlusions are fairly common and potentially dangerous. A process of the AE prediction model 24 that detects 72 a rise in downstream pressure and a change 68 in vital signs suggests that the vein is being occluded 66 and the patient is no longer receiving a critical infusion. In such case, the dongle 10 detects 70 rising backflow pressure. Thus, the system 2 can determine if the increasing backflow pressure is preventing a patient from receiving the required therapy. If so, a notification from the AE prediction model 24 is sent 74 to the practitioner. The practitioner, recognizing the potential for vein occlusion but not yet at a critical stage, can proactively enhance the model's monitoring capabilities.

By increasing the frequency 56 of data collection from the PVC system 34, the model 24 gains a more granular view of the evolving situation, enabling earlier detection of subtle changes that might precede a full occlusion. Thus, disclosed methods and systems extract critical information from both inside and outside current transmission protocols to create new features and models that provide much improved adverse event prevention, e.g., preventing occlusions that arise during IV administration of fluids and/or medications.

Advantageously, the adverse event prediction model 24 provides practitioners with significantly more information at their disposal to determine if a patient is going to have an AE. The information can help practitioners identify a precursor to a downstream occlusion. For example, a practitioner would want to be aware of increasing backflow pressure because that might indicate a vein is getting compromised, which would cause a patient not to receive the full effect of a drug. IV administration of morphine is an example. If the vein is getting to a point where it isn't receiving the drug anymore (and thus is spilling out everywhere-infiltration and/or extravasation), the medical practitioner would see the results of the “lack of morphine” shown in the vital signs. All AEs will have some form of correlation back to vital signs but it is the relationship of these auxiliary measurements (that are not presented) to vital signs that will likely become the AE's precursor.

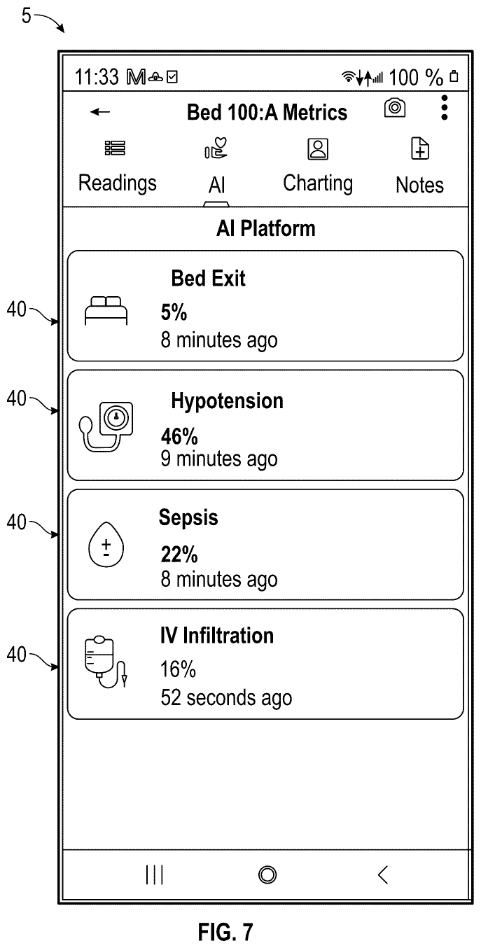

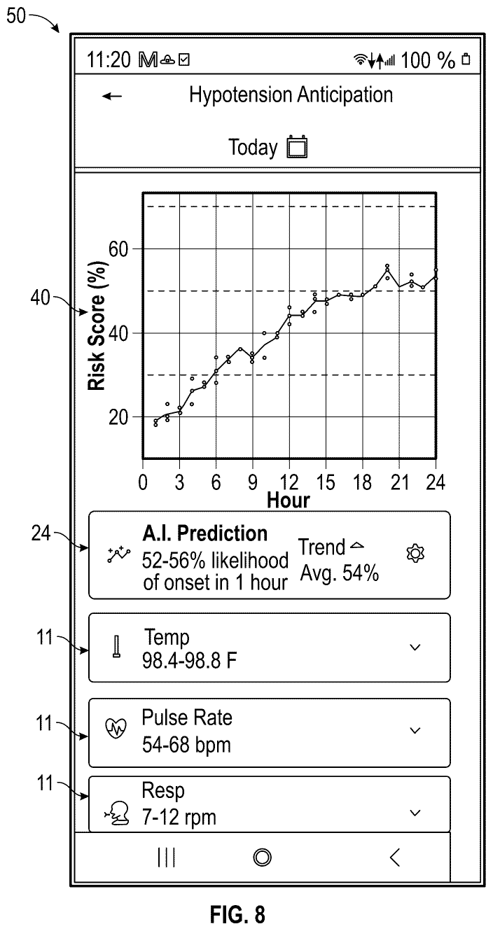

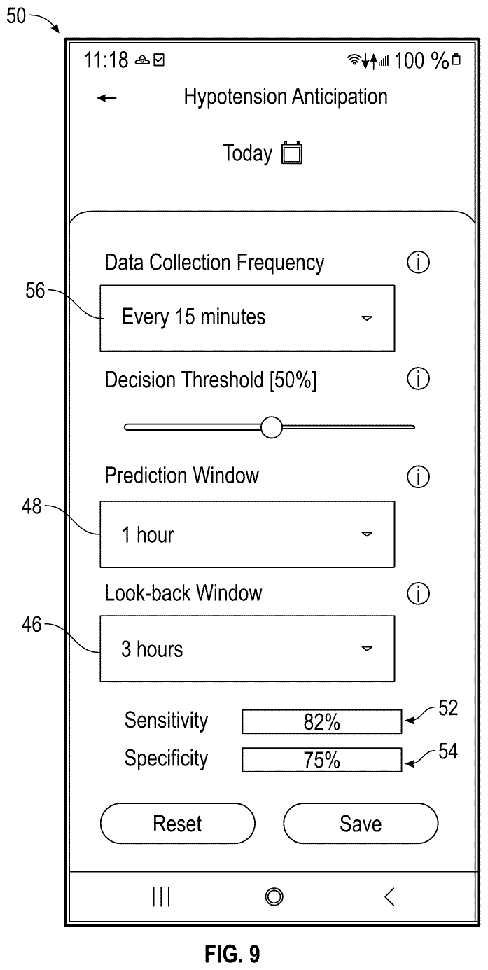

Referring to FIGS. 5 and 7-9, exemplary methods and systems of creating AI processes for AE prediction allow for real time tuning 38 of the model's characteristics. This is another advantage of deploying the AE prediction model 24 in the cloud 20. As shown in FIGS. 7 and 8, in exemplary embodiments the AE prediction model 24 presents to a medical practitioner, e.g., via the practitioner's mobile device 50 a risk score representing an assessment 40 of the level of risk that a patient will experience an adverse event. The practitioner may accept 42 the output and take 44 the necessary action. Alternatively, as best seen in FIG. 9, the practitioner may choose to tune 38 the model's data frequency 56 characteristics if he/she does not accept 42 the output, that is, if the practitioner believes that the model's default settings do not match the patient's characteristics. These characteristics can be adjusted using the cog wheel 58 on the user interface.

For example, the practitioner might believe that the patient is more likely to experience an AE after a physical assessment of the patient. Though the prescribed risk score 40 seems normal at that point in time, the nurse believes that physiological decline is still likely. As a result, the practitioner rejects the current risk score to have the model consider data on a more granular scale, e.g., from every thirty minutes to every five minutes. This change is received by the system, and a command from the network hub 16 changes the hardware attachment's read frequency to match the granularity increase.

Referring to FIG. 9, practitioners also can tune the AE prediction model's performance by modifying its risk threshold. This threshold represents the probability at which the AE prediction model 24 determines an adverse event is likely to occur, which subsequently triggers an alert on the system. In exemplary embodiments, the risk threshold is initially set to a value that the AE prediction model 24 believes provides the best balance between sensitivity 52 and specificity 54 based on the data the model was trained on initially. Sensitivity 52, in this context, refers to the model's ability to correctly identify instances where an adverse event is truly occurring. A highly sensitive model is designed to err on the side of caution, prioritizing early detection even if it means occasionally raising false alarms and contributing to alarm fatigue.

Specificity 54, on the other hand, refers to the ability of the AE prediction model 24 to correctly identify instances where no adverse event is actually taking place. A model with high specificity aims to minimize false alarms, providing alerts only when there is a strong indication of a genuine risk. However, the practitioner can adjust this risk threshold based on a specific patient's condition and risk factors to balance between these two performance metrics. Lowering the threshold increases the sensitivity of the AE prediction model 24, making it more likely to detect potential adverse events early on, but also increasing the likelihood of false alarms. Conversely, raising the threshold increases the model's specificity, reducing the number of false alarms but potentially delaying the detection of true adverse events.

The choice of risk threshold depends on various factors, including the patient's individual risk profile, the severity of the potential adverse event, and the clinical context. For instance, in a critically ill patient with a high risk of complications, a lower threshold might be appropriate to prioritize early detection and intervention. In contrast, for a stable patient with a low risk profile, a higher threshold might be preferred to avoid unnecessary alarms and interventions. By adjusting the risk threshold, the practitioner can tailor the performance of the AE prediction model 24 to the specific needs and circumstances of each patient, ensuring timely and accurate alerts while minimizing unnecessary disruptions to care.

Referring again to FIG. 9, in addition to the ability to adjust the risk threshold, a practitioner also can elect to adjust the lookback 46 and prediction windows 48 of the AE prediction model 24 to offer an additional layer of flexibility in tailoring the monitoring system to individual patient needs. The lookback window 46 functions as the model's memory, determining the timeframe of past data it considers when making predictions. A longer lookback window 46 provides a broader context, potentially revealing subtle long-term trends, but may also dilute the impact of recent, acute changes. Conversely, a shorter lookback window 46 focuses on the most immediate data, which is crucial for rapidly changing conditions but potentially misses slower-developing patterns.

Prediction windows 48 impact how far into the future the model attempts to forecast. A longer prediction window 48 can offer valuable lead time for proactive interventions, but it also increases the risk of inaccurate predictions due to the inherent uncertainties in forecasting complex physiological processes. A shorter prediction window 48 might provide more reliable short-term forecasts but could miss early warning signs of impending adverse events. These are additional examples of various characteristics a practitioner may change depending on their physical assessment of the patient following an initial alert.

The optimal choice for both lookback 46 and prediction windows 48 depends on the specific patient's condition and risk profile. For instance, a patient in critical care with rapidly fluctuating vital signs might benefit from a shorter lookback window 46 to prioritize the most recent data and a longer prediction window 48 to anticipate potential complications. In contrast, a stable patient might be better served by a longer lookback window 46 to establish a comprehensive baseline and a shorter prediction window 48 for timely alerts.

Advantageously, the adaptability of the AI platform 5 extends far beyond adjusting timeframes. The platform's innovative feature engineering capabilities enable it to create unique, patient-specific metrics that capture the complex interplay between various physiological parameters and the drug infusion process. By analyzing raw device data 12 alongside processed vital signs data 11, the system 2 can derive novel features that provide a more nuanced understanding of a patient's condition than traditional monitoring methods. Additionally, the system 2 can incorporate data from other medical instruments, such as infusion pumps 36 and patient beds 30, to create a more holistic view of the patient's physiological state. By incorporating these and other custom features, the AE prediction model 24 can better discern subtle patterns and anomalies that might otherwise go unnoticed, leading to earlier intervention and potentially preventing adverse events.

Thus, it is seen that systems and methods of creating artificial intelligence processes for predicting adverse events in a patient and combined systems and methods of extracting medical data to train an AI-based adverse event prediction model and predicting adverse events in patients are provided. It should be understood that any of the foregoing configurations and specialized components may be interchangeably used with any of the systems of the preceding embodiments. Although illustrative embodiments are described hereinabove, it will be evident to one skilled in the art that various changes and modifications may be made therein without departing from the disclosure. It is intended in the appended claims to cover all such changes and modifications that fall within the true spirit and scope of the disclosure.

While the disclosed systems and devices have been described in terms of what are presently considered to be the most practical exemplary embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A computer-implemented method of training an artificial intelligence process for predicting adverse events in a patient, comprising:
extracting processed vital signs medical data from a server in communication with at least one vital signs monitor;
extracting raw non-vital signs medical data from at least two different medical instruments, including extracting auxiliary medical data, including metadata, which one or more of the at least two different medical instruments do not have the ability to stream, which are outside of common transmission protocols, and which are not displayed by one or more of the at least two different medical instruments by identifying analog sensor data and deconstructing digitized communication packets;
combining the processed vital signs medical data and the raw non-vital signs medical data; and
providing the combined processed vital signs medical data and raw non-vital signs medical data to an artificial intelligence platform to train an AI-driven adverse event prediction model on the combined processed vital signs medical data and raw non-vital signs medical data;
the AI-driven adverse event prediction model generating novel features and composite features from the combined processed vital signs medical data and raw non-vital signs medical data to identify patterns indicative of potential adverse events.

2. The computer-implemented method of claim 1 further comprising:
continuing to extract the processed vital signs medical data from the server;
continuing to extract the raw non-vital signs medical data from the at least two different medical instruments by identifying analog sensor data and deconstructing digitized communication packets;
combining the processed vital signs medical data and the raw non-vital signs medical data; and
automatically providing the combined processed vital signs medical data and raw non-vital signs medical data to an artificial intelligence platform to input into the AI-driven adverse event prediction model;
wherein the AI-driven adverse event prediction model employs machine learning techniques to continuously improve predictive accuracy based on new input data and outcome feedback.

3. The computer-implemented method of claim 2 wherein the AI-driven adverse event prediction model analyzes the combined processed vital signs medical data and raw non-vital signs medical data for potential onset of at least one adverse event including identifying adverse event precursors in the combined processed vital signs medical data and the raw non-vital signs medical data.

4. The computer-implemented method of claim 3 wherein the AI-driven adverse event prediction model provides an assessment of a level of risk that a patient will experience an adverse event and a timeframe within which the patient will experience the adverse event.

5. The computer-implemented method of claim 1 wherein the extracting raw non-vital signs medical data from the at least two different medical instruments generates a continual stream of the raw non-vital signs medical data from the at least two different medical instruments that otherwise do not have an independent ability to continually stream the raw non-vital signs medical data.

6. The computer-implemented method of claim 1 wherein the artificial intelligence platform is cloud-based.

7. The computer-implemented method of claim 4 further comprising tuning the adverse event prediction model in real time while assessing a patient if default settings of the adverse event prediction model do not match the patient's characteristics.

8. The computer-implemented method of claim 7 wherein the tuning comprises one or more of: modifying data frequency characteristics, modifying sensitivity and specificity by adjusting a risk threshold, or modifying a lookback window and a prediction window.

9-10. (canceled)

11. An artificial intelligence system for predicting adverse events in patients, comprising:
at least one dongle configured to attach to at least one medical instrument such that the at least one dongle extracts raw non-vital signs medical data from at least two different medical instruments, including extracting auxiliary medical data which one or more of the at least two different medical instruments do not have the ability to stream, which are outside of common transmission protocols, and which are not displayed by one or more of the at least two different medical instruments, by identifying analog sensor data and deconstructing digitized communication packets;
a microcomputer in communication with the at least one dongle, the microcomputer receiving the raw non-vital signs medical data from the at least one dongle;
a cloud database in communication with the microcomputer and a client server, the client server receiving processed vital signs medical data from at least one vital signs monitor, the cloud database receiving the raw non-vital signs medical data from the microcomputer and the processed vital signs medical data from the client server; and
an artificial intelligence platform in communication with the cloud database, the artificial intelligence platform receiving the raw non-vital signs medical data and processed vital signs medical data from the cloud database and inputting the raw non-vital signs medical data and the processed vital signs medical data into an AI-driven adverse event prediction model;
the AI-driven adverse event prediction model generating novel features and composite features from the processed vital signs medical data and the raw non-vital signs medical data to identify patterns indicative of potential adverse events;
wherein the AI-driven adverse event prediction model can be tuned by a medical practitioner in real time while assessing a patient if default settings of the adverse event prediction model do not match the patient's characteristics, the tuning comprising one or more of: modifying data frequency characteristics, adjusting a risk threshold, or modifying a lookback window and a prediction window.

12. The artificial intelligence system of claim 11 wherein one or more of the at least two different medical instruments is a load sensor or a set of load sensors for a patient's bed and the processed vital signs medical data are one or more of: pulse rate, pulse rate variability, respiratory rate, blood pressure, and blood oxygen.

13. The artificial intelligence system of claim 11 wherein one or more of the at least two different medical instruments is an infusion pump and the raw non-vital signs medical data include backflow pressure.

14. The artificial intelligence system of claim 11 wherein the at least one dongle continues to extract raw non-vital signs medical data from the at least two different medical instruments by identifying analog sensor data and deconstructing digitized communication packets;
wherein the microcomputer continues to receive the raw non-vital signs medical data from the at least one dongle;
wherein the client server continues to receive the processed vital signs medical data from the at least one vital signs monitor and the cloud database continues to receive the raw non-vital signs medical data from the microcomputer and the processed vital signs medical data from the client server;
wherein the artificial intelligence platform continues to receive the raw non-vital signs medical data and the processed vital signs medical data from the cloud database and automatically inputs the raw non-vital signs medical data and the processed vital signs medical data into the AI-driven adverse event prediction model; and
wherein the AI-driven adverse event prediction model employs machine learning techniques to continuously improve predictive accuracy based on new input data and outcome feedback.

15. The artificial intelligence system of claim 14 wherein the AI-driven adverse event prediction model analyzes the processed vital signs medical data and the raw non-vital signs medical data for potential onset of at least one adverse event including identifying adverse event precursors in the processed vital signs medical data and the raw non-vital signs medical data.

16. The artificial intelligence system of claim 15 wherein the AI-driven adverse event prediction model provides an assessment of a level of risk that a patient will experience an adverse event and a timeframe within which the patient will experience the adverse event.

17. The artificial intelligence system of claim 11 wherein the at least one dongle generates a continual stream of the raw non-vital signs medical data from one or more of the at least two different medical instruments.

18. A combined system of extracting medical data to train an AI-based adverse event prediction model and predicting adverse events in patients, comprising:
at least one dongle configured to attach to at least one medical instrument such that the at least one dongle extracts raw non-vital signs medical data from the at least one medical instrument, including extracting auxiliary medical data which are not displayed by the at least one medical instrument by identifying analog sensor data and deconstructing digitized communication packets;
a microcomputer in communication with the at least one dongle, the microcomputer receiving the raw non-vital signs medical data from the at least one dongle; and
a cloud database in communication with the microcomputer and a client server, the client server receiving processed vital signs medical data from at least one vital signs monitor, the cloud database receiving the raw non-vital signs medical data from the microcomputer and the processed vital signs medical data from the client server; and
an artificial intelligence platform in communication with the cloud database, the artificial intelligence platform receiving the raw non-vital signs medical data and the processed vital signs medical data from the cloud database and inputting the raw non-vital signs medical data and the processed vital signs medical data into an AI-driven adverse event prediction model;
the AI-driven adverse event prediction model generating novel features and composite features from the processed vital signs medical data and the raw non-vital signs medical data to identify patterns indicative of potential adverse events, the composite features including a restlessness score factoring in one or more of: movement frequency, vital sign changes, volume of IV fluid infused, and time of day;
wherein the at least one dongle continues to extract raw non-vital signs medical data from the at least one medical instrument, including continuing to extract the auxiliary medical data which are not displayed by the at least one medical instrument;
wherein the client server continues to receive processed vital signs medical data from the at least one vital signs monitor and the cloud database continues to receive the raw non-vital signs medical data from the microcomputer and the processed vital signs medical data from the client server;
wherein the artificial intelligence platform continues to receive the raw non-vital signs medical data and the processed vital signs medical data from the cloud database and automatically inputs the raw non-vital signs medical data and the processed vital signs medical data into the AI-driven adverse event prediction; and
wherein the AI-driven adverse event prediction model employs machine learning techniques to continuously improve predictive accuracy based on new input data and outcome feedback.

19. The combined system of claim 18 wherein the AI-driven adverse event prediction model provides an assessment of a level of risk that a patient will experience an adverse event.

20. The combined system of claim 18 further comprising a user interface whereby a medical practitioner can tune the adverse event prediction model in real time while assessing a patient if default settings of the adverse event prediction model do not match the patient's characteristics, the tuning comprising one or more of:
modifying data frequency characteristics, adjusting a risk threshold, or modifying a lookback window and a prediction window.

21. The computer-implemented method of claim 2 wherein the at least two different medical instruments comprise an infusion pump and a load sensor for a patient's bed.

22. The computer-implemented method of claim 2 further comprising tuning the adverse event prediction model in real time via a user interface while assessing a patient if default settings of the adverse event prediction model do not match the patient's characteristics.
